(19) United States
(12) Patent Application Publication
(10) Pub. No.: US 2012/0220018 A1
(43) Pub. Date: Aug. 30, 2012

(54) Method for Nutrient Pre-Loading of Microbial Cells

(75) Inventor: David A. Hazlebeck, EI Cajon, CA (US)

(73) Assignee: General Atomics, San Diego, CA (US)

(21) Appl. No.: 13/468,722

(22) Filed: May 10, 2012

Related U.S. Application Data
(62) Division of application No. 12/491,039, filed on Jun. 24, 2009.

(51) Int. Cl. C12N 1/14; C12N 1/12

(52) U.S. Cl. 435/254.1; 435/257.1

(57) ABSTRACT

A method is provided for supporting the growth of selected microbial cells and for obstructing the growth of contaminants in a non-sterile system. In the method, the microbial cells are pre-loaded with a surplus amount of a chosen nutrient, such as phosphorus, other macronutrients, or micronutrients. Further, the chosen nutrient is greatly reduced, or eliminated, from the non-sterile system. Thereafter, the pre-loaded selected microbial cells are introduced into the non-sterile system. In the non-sterile system, the selected microbial cells rely on the surplus amount of the chosen nutrient to survive and grow. At the same time, contaminants such as non-selected microbial strains and bacteria starve from a lack of the chosen nutrient in the non-sterile system.

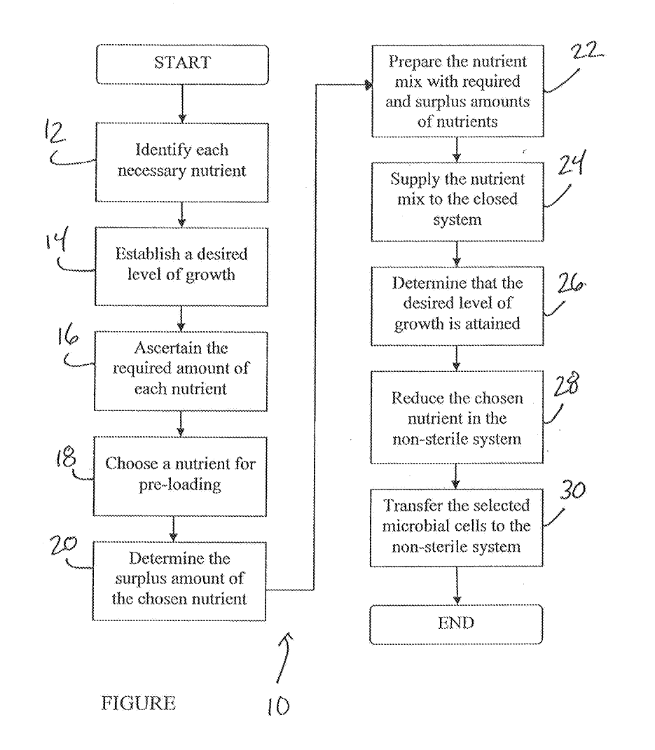

This application is a divisional of application Ser. No. 12/491,039, filed Jun. 24, 2009, which is currently pending. The contents of Application Ser. No. 12/491,039 are incorporated herein by reference.

### FIELD OF THE INVENTION

The present invention pertains generally to methods for growing microbial cells, such as microalgae, fungi, or cyanobacteria cells. More particularly, the present invention pertains to the treatment of selected microbial cells to maximize their growth while obstructing the growth of contaminants. The present invention is particularly, but not exclusively, useful as a method for pre-loading selected microbial cells with a surplus amount of a chosen nutrient before growing the selected microbial cells in a non-sterile system devoid of the chosen nutrient.

### BACKGROUND OF THE INVENTION

As worldwide petroleum deposits decrease, there is rising concern over petroleum shortages and the costs that are associated with the production of hydrocarbon products. As a result, alternatives to products that are currently processed from petroleum are being investigated. In this effort, biofuel such as biodiesel has been identified as a possible alternative to petroleum-based transportation fuels. In general, a biodiesel is a fuel comprised of mono-alkyl esters of long chain fatty acids derived from plant oils or animal fats. In industrial practice, biodiesel is created when plant oils or animal fats are reacted with an alcohol, such as methanol.

For plant-derived biofuel, solar energy is first transformed into chemical energy through photosynthesis. The chemical energy is then refined into a usable fuel. Currently, the process involved in creating biofuel from plants is expensive relative to the process of extracting and refining petroleum. It is possible, however, that the cost of processing a plant-derived biofuel could be reduced by maximizing the rate of growth of the plant source.

Microalgae and cyanobacteria are known to be some of the most efficient plants for converting solar energy into cell growth, they are of particular interest as biofuel sources. Importantly, the use of microalgae or cyanobacteria as a biofuel source presents no exceptional problems, i.e., biofuel can be processed from microalgae or cyanobacteria as easily as from land-based plants.

While microalgae and cyanobacteria can efficiently transform solar energy into chemical energy via a high rate of cell growth, it has been difficult to create environments in which cell growth rates are optimized. Currently, the production of biofuel from algae is limited by a failure to maximize algae cell growth. Specifically, the conditions necessary to facilitate a fast growth rate for algae cells in large-scale operations have been found to be expensive to create. For instance, while providing high rates of algae cell growth, closed sterile environments such as inoculant tanks and controlled bioreactors are expensive to maintain and limited in scale. On the other hand, non-sterile large-scale closed systems and outdoor large-scale open systems, such as open runways, are plagued by contaminant organisms which fight the selected algae cells for nutrients and sunlight and reduce the rate of algae cell growth. Specifically, these contaminants include non-selected, i.e., “weed”, algae and bacteria. Until now, it has been virtually impossible to prevent contaminant organisms from causing microbial instability and reduce selected algae cell growth rates in open systems.

Also, microalgae, fungi, and cyanobacteria can be grown heterotrophically or mixotrophically to produce materials for biofuel production. For example, microalgae can be grown on cellulosic and hemicellulosic sugars to produce lipids. However, the cost of lipid production is too high if a sterile environment is implemented. On the other hand, bacteria will out-compete the microalgae if a non-sterile system is used. Until now, sterile systems have been required for heterotrophic or mixotrophic conversion of sugars to biofuels.

In light of the above, it is an object of the present invention to provide a method for maximizing the cell growth of selected microbial cells in a non-sterile system. Another object of the present invention is to provide a method for pre-loading selected microbial cells with at least one chosen nutrient in order to prepare the selected microbial cells for growth in a system lacking the chosen nutrient. Still another object of the present invention is to provide a method and system for growing selected microbial cells in a system in which contaminants are starved. Another object of the present invention is to initially pre-load selected microbial cells with a chosen nutrient in a medium and thereafter to eliminate the chosen nutrient from the medium to prevent the growth of contaminants. Yet another object of the present invention is to provide a system and method for growing selected microbial cells that is simple to implement, easy to use, and comparatively cost effective.

### SUMMARY OF THE INVENTION

In accordance with the present invention, a system is provided for growing selected microbial cells in a medium and for preventing the growth of contaminants in the medium. Structurally, the system includes a first closed reactor system and a second non-sterile reactor system. For purposes of the present invention, the closed reactor can be a continuous flow reactor, semi-batch reactor, batch reactor or combination of these. The closed reactor system contains a medium with a nutrient mix. For the present invention, the second non-sterile reactor system receives an effluence containing microbial cells from the first closed reactor. Further, the second stage reactor includes a conduit for continuously moving the effluence downstream. Preferably, the second reactor system is a plug flow reactor system in the form of an open raceway for photosynthetic growth. In certain embodiments, the second reactor system is a semi-batch reactor system for heterotrophic growth.

In general, the method of the present invention involves preparation of the selected microbial cells in the closed reactor to withstand non-life-sustaining conditions in the non-sterile reactor system. Importantly, the non-life-sustaining conditions are preserved in the non-sterile reactor system to prevent the growth of contaminant organisms. Further, the non-life-sustaining conditions in the non-sterile reactor system are overcome by the propensity of the selected microbial cells to take in surplus amounts of certain nutrients. For instance, a specific strain of microalgae may take in ten to twelve times as much phosphorus as would be required for normal growth. Therefore, if pre-loaded with a surplus of phosphorus, this strain of microalgae could survive in an open reactor which lacks phosphorus for quite some time.

In the method of the present invention, the closed reactor is provided and a nutrient mix is prepared. While the nutrient mix includes the required amount of each nutrient that is necessary for a desired level of growth of the selected microbial cells, it further includes a surplus amount of a chosen nutrient that exceeds the respective required amount of the chosen nutrient. After preparation of the nutrient mix, it is fed into the closed reactor and the selected microbial cells are allowed to grow. Thus, as a sterile, closed reactor, no contaminant organisms compete with the selected microbial cells for the nutrient mix. As the selected microbial cells grow to the desired level, they store the surplus amount of the chosen nutrient.

When the selected microbial cells have reached the desired level of growth, the non-sterile reactor system is prepared to receive them. Specifically, the chosen nutrient is greatly reduced, or eliminated, from the non-sterile reactor system. Further, the other nutrients required for growth by the selected microbial cells are provided in the non-sterile reactor system. After the non-sterile reactor system is readied, the selected microbial cells are transferred from the closed reactor to the non-sterile reactor system. When the selected microbial cells are introduced into the non-sterile reactor system, they utilize the stored surplus amount of the chosen nutrient and the other nutrients present in the non-sterile reactor system to grow. Because the non-sterile reactor system lacks the chosen nutrient, contaminant organisms cannot grow and compete with the selected microbial cells for nutrients or sunlight. As a result, the growth rate of the selected microbial cells in the non-sterile reactor system is maximized.

Typically, the chosen nutrient is a macronutrient such as phosphorus, nitrogen, calcium, or potassium. Alternatively, the chosen nutrient may be a plurality of micronutrients that are unlikely to be unintentionally introduced into the non-sterile reactor system.

### BRIEF DESCRIPTION OF THE DRAWINGS

The novel features of this invention, as well as the invention itself, both as to its structure and its operation, will be best understood from the accompanying drawing, taken in conjunction with the accompanying description, in which the FIGURE is a schematic illustrating the steps of the method in an embodiment of the present invention.

### DESCRIPTION OF THE PREFERRED EMBODIMENTS

In the Figure, it can be seen that the method, generally designated by reference number 10, can be considered to begin with the step of identifying each nutrient necessary for growth of selected microbial cells (action block 12). Generally, these nutrients include macronutrients such as phosphorus, nitrogen, calcium, sulfur, carbon, hydrogen, oxygen, iron, magnesium and potassium and micronutrients which may include silicon, chloride, sodium, copper, zinc, and manganese. However, the selected microbial cells may require fewer or more nutrients than those listed here.

At action block 14, a desired level of growth is established for the microbial cells. Typically, the desired level of growth will be related to the original concentration of the microbial cells and to the size of the closed system used to grow the microbial cells.

With the desired level of growth in mind, the amount of each identified nutrient required to support such growth can be ascertained (action block 16). Further, at action block 18, at least one nutrient is chosen from the identified nutrients for pre-loading the microbial cells. Specifically, microbial cells can store an excess amount of certain nutrients. Therefore, one or more of these certain nutrients is chosen to be supplied in excess to the microbial cells in the closed system.

At action block 20, the surplus amount of the chosen nutrient which will be supplied to the microbial cells in the closed system is determined. Preferably, this step requires knowledge of how much excess amount of the chosen nutrient the microbial cells may store. For example, if the microbial cells will store fifteen times as much phosphorus as is required for growth to the desired level, then the surplus amount of phosphorus may be determined to be ten to twelve times as much as the required amount of phosphorus.

After the required amounts of necessary nutrients are ascertained and the surplus amount of the chosen nutrient is determined, the nutrients are prepared in a nutrient mix (action block 22). In action block 24, the nutrient mix is supplied to the closed system to support growth of the microbial cells to the desired level. While this mix may be prepared and provided in a single batch, it is also envisioned that the nutrients in the mix may be separately stored and provided to the microbial cells. For instance, the nutrients may be provided to the medium in the closed system as needed by the growing microbial cells.

While the microbial cells grow in the closed system, they are monitored to determine when they have attained the desired level of growth and have stored the surplus amount of the chosen nutrient (action block 26). In certain embodiments, this step may be performed by measuring the duration of time after the nutrient mix is fed into the closed reactor. For such an embodiment, the desired level of growth and storage of the surplus nutrient is considered to be attained after the duration of time equals a pre-determined value, such as one day. In other embodiments, the medium in the closed reactor is monitored and the microbial cells are considered to have attained the desired level of growth and surplus nutrient storage when the amount of at least one nutrient in the closed reactor equals a pre-determined value, such as zero.

While the microbial cells are pre-loaded with the surplus amount of the chosen nutrient in the closed reactor, a non-sterile system, such as an outdoor runway, is prepared for continued growth of the microbial cells. Specifically, at action block 28, the chosen pre-loaded nutrient is eliminated, or reduced to levels that cannot sustain life, in the non-sterile system. Thereafter, the microbial cells are transferred from the closed reactor to the non-sterile system (action block 30). Because the selected microbial cells are preloaded with the chosen nutrient, the absence of the chosen nutrient in the non-sterile system does not affect the ability of the selected microbial cells to thrive. Specifically, the selected microbial cells draw on their stores of surplus amounts of the chosen nutrient. Further, the non-sterile system contains sufficient amounts of the other nutrients to support continued growth of the selected microbial cells while the stores of the chosen nutrient are drawn on. Importantly, growth of contaminant microorganisms such as bacteria and non-selected microbial species is not supported in the non-sterile system. Because the chosen nutrient is absent from the non-sterile system, the contaminants cannot thrive and, therefore, do not compete with the selected microbial cells for the nutrients provided in the non-sterile system. In other words, the contaminants starve from a lack of the chosen nutrient in the non-sterile system.

While the particular Method for Nutrient Pre-loading of Microbial Cells as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A method for growing selected microbial cells in a non-sterile system comprising the steps of:
identifying each nutrient needed for growth of the selected microbial cells;
establishing a desired level of growth of the selected microbial cells;
ascertaining a required amount of each identified nutrient to support the desired level of growth of the selected microbial cells;
choosing a nutrient from the identified nutrients for microbial cell preloading;
determining a surplus amount of the chosen nutrient, with the surplus amount exceeding the required amount of the chose nutrient;
supplying the surplus amount of the chosen nutrient and the required amounts of the identified nutrients to the selected microbial cells, wherein the selected microbial cells achieve the desired level of growth and wherein the selected microbial cells store the surplus amount of the chosen nutrient;
reducing the chosen nutrient in the non-sterile system; and
transferring the selected microbial cells to the non-sterile system, where the selected microbial cells rely on the stored surplus amount of the chosen nutrient for growth.

2. A method as recited in claim 1 wherein contaminants in the non-sterile system starve from a lack of the chosen nutrient in the non-sterile system.

3. A method as recited in claim 1 wherein the supplying step is performed in a controlled closed reactor.

4. A method as recited in claim 1 wherein the reducing step is accomplished by eliminating the chosen nutrient from the non-sterile system.
